(19) United States
(12) Patent Application Publication
(10) Pub. No.: US 2014/0349281 A1
(43) Pub. Date: Nov. 27, 2014

(54) System and Method for Dispensing Barcoded Solutions

(71) Applicant: Sunpower Technologies LLC, San Marcos, CA (US)

(72) Inventor: Travis Jennings, San Diego, CA (US)

(73) Assignee: Sunpower Technologies LLC, San Marcos, CA (US)

(21) Appl. No.: 13/899,814

(22) Filed: May 22, 2013

(51) Int. Cl. C12Q 1/68

(52) U.S. Cl. CPC C12Q 1/6895
USPC 435/6.1; 435/287.2

(57) ABSTRACT

The present disclosure provides a system capable of dispensing DNA encoding solutions at controllable flow rates and in uniform patterns, allowing sufficient DNA to be deposited onto a substrate for subsequent detection and identification of the substrate. The disclosed dispensing system may also include a feedback mechanism capable of validating that sufficient DNA has been applied to a substrate so that a barcode may be generated, detected, and identified at a later time.

### BACKGROUND

1. Field of the Disclosure

The present disclosure relates generally to biological material ID systems, and more particularly DNA barcode dispensing systems.

2. Background Information

In many cases the movement and distribution of biological materials, such as plants, crops and seeds, has to be tracked and controlled, for various reasons.

In research facilities and in the industry, medical and healthcare industries for example, biological materials are used to derived substances that may be applied in therapeutic treatment of illnesses, but illegal, toxic substances can also be derived from these biological materials, which raises the need to track and control the movement of the biological materials.

There are few methods for identifying legal biological material products from illegal varieties; however, there are certain methods that may modify these biological material products or their production, which may be considered neither convenient nor accurate, and may represent a high cost for several regulation entities. For example, the use of genetic engineering may innately modify the plant in a very fundamental form.

There is therefore a need to be able to distinguish authorized biological materials from common, illegal toxic varieties of biological materials; a new method may be applied to perform the identification of legal and illegal biological materials with more accuracy and lower cost.

A new method may be applied to perform the identification of biological materials with more accuracy and lower cost, but there still is a need for a device capable of accurately applying encoding solutions to biological materials in a controllable and verifiable manner.

### SUMMARY

Systems capable of dispensing DNA encoding solutions at controllable flow rates and in uniform patterns are disclosed. These systems may allow sufficient DNA oligomers to be deposited onto a substrate for subsequent detection and identification of the substrate. The disclosed dispensing system may also include a feedback mechanism capable of validating that sufficient DNA has been applied to a substrate so that a barcode may be generated, detected, and identified at a later time.

The encoding may be done directly depositing a solution including one or more oligonucleotides onto the product. At growth facilities, biological materials may be marked with an encoding solution of synthetic DNA oligomers which may be used to encode for specific properties related to the biological materials and which may be used to validate the authenticity of the biological materials later. For the downstream readout and identification technology to operate correctly, there must be a minimum quantity of DNA oligomers applied to the biological materials, which must be applied in a manner such that the portion of the biological materials which has been sprayed may be easily discovered.

The disclosed dispensing device may include a DNA reservoir, a spraying module, and a feedback module.

The DNA reservoir may be an enclosure designed to hold suitable amounts of encoding solutions, which may include coding strands and quantification strands of DNA. The coding strands may be synthetic DNA oligomers, in most cases between about 20 and about 50 base pairs in length. Encoding solution may include between about 2 and about 20 different coding strands, where each coding strand may be capable of encoding a particular characteristic of the biological materials.

Quantification strands may be synthetic DNA oligomers conjugated with a fluorescent dye molecule. There could be one or more dye molecules conjugated with a single quantification strand. Quantification strands may provide a fluorescent marker within the encoding solution, which may be used to accurately estimate the quantity of coding strands deposited on a sample or a substrate.

The spraying module may include a nozzle capable of dispensing the DNA solution in a flat jet stream. Suitable nozzle designs include hemispherical inlet nozzles and V-notch nozzles, among others. These nozzles may be capable of atomizing in a controllable flat jet spray so that the solution may be focused for precise delivery and avoid waste.

The feedback module may include a laser module and an optical detection module. The feedback module may be capable of monitoring the dispensing of the encoding solution by detecting the DNA after it is deposited onto a suitable substrate. Using the laser module, the amount of fluorescent dye deposited onto a substrate may be quantified by measuring the fluorescence intensity from the dye. The laser beam, produced by laser module, may excite the fluorescent molecules attached to quantification strands and cause them to emit fluorescent light. The intensity of the light emitted by the fluorescent molecules may be directly proportional to the amount of dye deposited, which in turn may be directly proportional to the amount of coding strands deposited. Hence, by measuring the intensity of the fluorescent light, the amount of coding strands deposited may be approximated, allowing a consistent measurement of the amount of DNA oligomers deposited.

The disclosed dispensing device may be capable of applying encoding solutions to substrates in a controllable, verifiable, and reproducible manner.

In one embodiment, a dispensing device comprises a DNA reservoir configured to store encoding solution, wherein the encoding solution comprises coding strands of DNA that encode a characteristic about a plant and quantification strands of DNA comprising synthetic DNA oligomers conjugated with fluorescent dye molecules; and a spraying device configured to dispense the encoding solution on the plant; whereby the encoding solution is illuminated by a laser beam. The dispensing device may further comprise a feedback device comprising a laser beam generator configured to generate a laser beam that excites the fluorescent dye molecules and cause the fluorescent dye molecules to emit fluorescent light, wherein an intensity of fluorescent light emitted by the fluorescent dye molecules is directly proportional to an amount of coding strands of DNA deposited on the plant; and an optical light detector configured to measure the intensity of the fluorescent light emitted by the fluorescent molecules and determine whether the intensity of the fluorescent light exceeds a first threshold, thereby determining whether the amount of coding strands of DNA deposited on the plant exceeds a second threshold.

In another embodiment, a method for measuring a quantity of a solution deposited on a plant comprises focusing a laser beam, by a steering optic, at a part of the plant where an encoding solution has been deposited to excite the fluorescent dye molecules and cause the fluorescent dye molecules to emit fluorescent light, wherein the encoding solution comprises coding strands of DNA that encode a characteristic about the plant and quantification strands of DNA comprising synthetic DNA oligomers conjugated with fluorescent dye molecules, and wherein an intensity of fluorescent light emitted by the fluorescent dye molecules is directly proportional to an amount of coding strands of DNA deposited on the plant; collecting light by a collection and focusing optic; receiving the collected light by a photodetector; measuring the intensity of the fluorescent light by the photodetector; and determining whether the intensity of the received fluorescent light exceeds a threshold by the photodetector, thereby determining whether a sufficient amount of coding strands of DNA have been deposited on the plant.

Additional features and advantages of an embodiment will be set forth in the description which follows, and in part will be apparent from the description. The objectives and other advantages of the invention will be realized and attained by the structure particularly pointed out in the exemplary embodiments in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The present disclosure can be better understood by referring to the following figures. The components in the figures are not necessarily to scale, emphasis instead being placed upon illustrating the principles of the disclosure. In the figures, reference numerals designate corresponding parts throughout the different views.

FIG. 1 is a flowchart of a dispensing device, according to an embodiment.

FIG. 2 illustrates a feedback module, according to an embodiment.

### DETAILED DESCRIPTION

The present disclosure is here described in detail with reference to embodiments illustrated in the drawings, which form a part here. Other embodiments may be used and/or other changes may be made without departing from the spirit or scope of the present disclosure. The illustrative embodiments described in the detailed description are not meant to be limiting of the subject matter presented here.

### Definitions

As used here, the following terms may have the following definitions:

“DNA oligomers” refers to short single-stranded of deoxyribonucleic acid (DNA) formed by bounded molecules.

“Coding strands” refers to single-stranded sequences of DNA used in lateral flow tests to generate barcodes.

“Barcode” refers to a pattern that allows the identification or verification of a type of living being, based on a DNA sequence.

“Biological material” refers to substances containing genetic information from organisms of the Plantae kingdom, such as plants and seeds, capable of reproducing themselves or being reproduced in a biological system.

### Description of the Drawings

DNA oligomers may be used to encode specific characteristics of biological materials. After encoding, suitable known in the art tests may be used to decode the DNA for interpretation, creating readable barcodes or patterns that may be compared with a database to determine if the biological materials come from approved growth facilities.

The encoding may be done directly depositing required amounts of an encoding solution including one or more DNA oligomers onto the biological material.

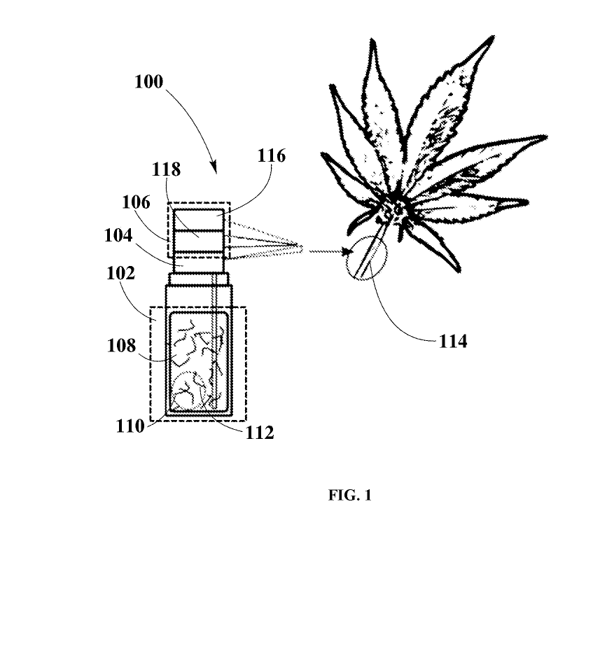

FIG. 1 shows a dispensing device 100. In one embodiment, dispensing device 100 may include DNA reservoir 102, spraying module 104, and feedback module 106. DNA reservoir 102 may be an enclosure designed to hold suitable amounts of solutions. DNA reservoir 102 may enclose encoding solution 108, which may include coding strands 110 and quantification strands 112 of DNA. Coding strands 110 may be synthetic DNA oligomers, in most cases between about 20 and about 50 base pairs in length. Encoding solution 108 may include between about 2 and about 20 different coding strands 110, where each coding strand 110 may be capable of encoding for a particular characteristic of the biological material. Each coding strand 110 may be present in encoding solution 108 in a concentration ranging from about 1 μM to about 50 μM. Quantification strands 112 may be synthetic DNA oligomers conjugated with a fluorescent dye molecule. Suitable fluorescent dyes may include Alexa Fluor® 647, Cy5, and DyLight 633, among others. The conjugation between the DNA strands and the fluorescent dye may take place between the 5′ and 3′ terminal ends of the DNA strand, or may be conjugated to an internal base. There could be one or more dye molecules conjugated with a single quantification strand 112.

Quantification strands 112 may provide a fluorescent marker in the encoding solution 108, which may be used to accurately estimate the quantity of coding strands 110 deposited on a sample or substrate.

Spraying module 104 may include a nozzle capable of dispensing the DNA solution in a flat jet stream. Suitable nozzle designs may include hemispherical inlet nozzles and V-notch nozzles, among others. These nozzles may be capable of atomizing in a controllable flat jet spray so that the solution may be focused for precise delivery and avoid waste. Spraying module 104 may be configured to deposit minimum of about 100 picomols of each DNA strand onto a detectable area of the substrate. This amounts to approximately 50 μL of a 2 μM solution.

Feedback module 106 may include a laser module 116 and an optical detection module 118. Feedback module 106 may be capable of monitoring the dispensing of encoding solution 108 by detecting the DNA after it is deposited onto a suitable substrate 114. Using laser module 116, the amount of fluorescent dye deposited onto a substrate may be quantified by measuring the fluorescence intensity from the dye. The laser beam, produced by laser module 116, may excite the fluorescent molecules attached to quantification strands 112 and cause them to emit fluorescent light. The intensity of the light emitted by the fluorescent molecules is directly proportional to the amount of dye deposited, which in turn is directly proportional to the amount of coding strands 110 deposited. Hence, by measuring the intensity of the fluorescent light, the amount of coding strands 110 deposited may be approximated. The information gathered by feedback module 106 may be translated into a format that an operator, operating the device, may be capable of recognizing and analyzing to determine if the required amount of encoding solution 108 has been deposited onto the desired substrate.

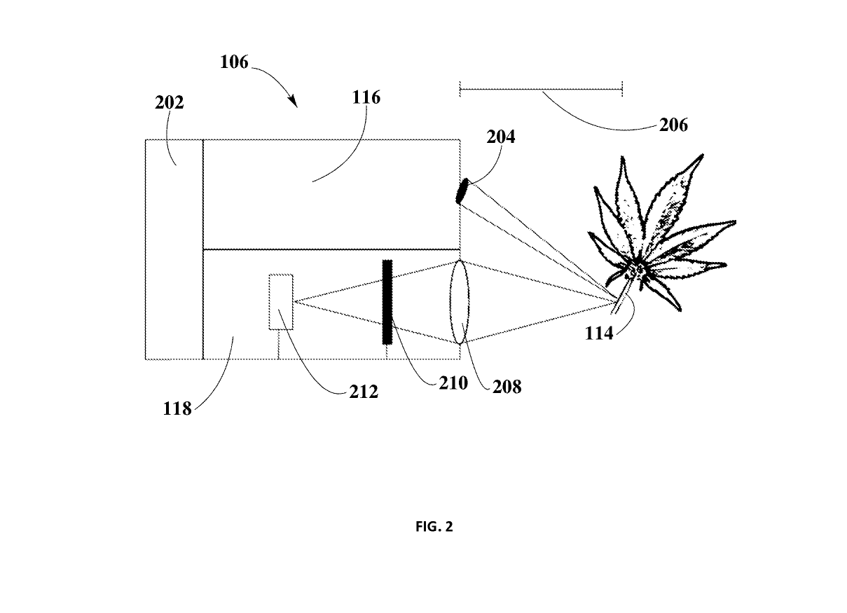

FIG. 2 illustrates a schematics of a feedback module 106 including a power supply 202, laser module 116, and optical detection module 118. Power supply 202 may include a battery pack or a plug-in power supply. Laser module 116 may include a common low power, lightweight laser system of common emission wavelengths, between 633 and 532 nm approximately and average power approximately between about 10 and about 50 mW. Exemplary laser modules 116 may include helium-neon lasers, among others. Laser module 116 may also include an output coupler or a steering optic 204 that may direct the beams emitted by laser module 116 so as to converge at or near the focal point of optical detection module 118. Laser module 116 may also assist the operator by providing aim guidance before spraying, which may ensure that encoding solution 108 is deposited onto the desired areas of substrate 114. For accurate deposition, dispensing device 100 may be positioned at a distance 206 from substrate 114 of about 0.5″ to about 2.5″, which may coincide with the focal distance of optical detection module 118.

Optical detection module 118 may include an f1 or f2 collection and focusing optic 208, an optical filter 210 and a suitable photodetector 212. Collection and focusing optics 208 may be protected by a splash guard to avoid optical interference. Collection and focusing optics 208 may be configured to collect all light at its focal point and then refocus the collected light into photodetector 212. Before reaching photodetector 212, the collected light may go through optical filter 210, which may be selected in such a way that it filters out the laser light and allows the fluorescent light to go through. Suitable optical filters 210 may include bandpass filters, notch filters and combinations thereof.

### EXAMPLES

In example #1 a biological material is encoded by depositing about 5 μL of encoding solution 108 onto the surface of the biological material. After harvest, the biological material is sprayed with a suitable encoding solution 108 including DNA oligomers, using a dispensing device 100, and left to dry. The encoding solution 108 used includes Alexa Fluor 647 fluorescent dye. A laser module 116 with a wavelength of about 633 nm is used as part of the feedback module 106. When laser module 116 illuminates the sprayed area, the dye particles are excited and emit fluorescent light of about 665 nm of wavelength. The intensity of the fluorescent light is measured by the optical detection module 118 and is determined that the amount of DNA oligomers deposited on the biological material is sufficient for the later identification of the biological material. Afterwards, the biological material is packed and shipped. At a storage facility, the biological material is identified, prior to be sold to a costumer. A sample from the biological material is taken, including the DNA oligomers (coding strands 110 and quantification strands 112). The sample is submerged in a suitable buffer solution, between about 1 and about 20 min. Then the mixture is deposited in a lateral flow test strip. As a result of the test a barcode may be generated. The barcode is read using a smartphone and is compared with a secure database of allowed results and the biological material is successfully identified.

In example #2 a cannabis plant is encoded by depositing about 5 μL of encoding solution onto the surface of the cannabis plant. After harvest, the cannabis plant is sprayed with a suitable encoding solution including DNA oligomers, using a dispensing device 100, and left to dry. The encoding solution used includes Alexa Fluor 647 fluorescent dye. A laser module 116 with a wavelength of about 633 nm is used as part of the feedback module 106. When laser module 116 illuminates the sprayed area, the dye particles are excited and emit fluorescent light of about 665 nm of wavelength. The intensity of the fluorescent light is measured by the optical detection module and it is determined that the amount of DNA oligomers deposited on the cannabis plant is sufficient for the later identification of the plant. Afterwards, the cannabis plant is packed and shipped. At a storage facility, the cannabis plant is identified, prior to be sold to a costumer. A sample from the cannabis plant is taken, including the DNA oligomers (coding strands 110 and quantification strands 112). The sample is submerged in a suitable buffer solution, between about 1 and about 20 min. Then the mixture is deposited in a lateral flow test strip. As a result of the test a barcode may be generated. The barcode is read using a smartphone and is compared with a secure database of allowed results. The cannabis plant is successfully identified and sold.

In example #3 a coca plant is encoded by depositing about 5 μL of encoding solution 108 onto the surface of the coca plant. After harvest, the coca plant is sprayed with a suitable encoding solution 108 including DNA oligomers, using a dispensing device 100, and left to dry. The encoding solution 108 used includes Alexa Fluor 647 fluorescent dye. A laser module 116 with a wavelength of about 633 nm is used as part of the feedback module 106. When laser module 116 illuminates the sprayed area, the dye particles are excited and emit fluorescent light of about 665 nm of wavelength. The intensity of the fluorescent light is measured by the optical detection module and it is determined that the amount of DNA oligomers deposited on the coca plant is sufficient for the later identification of the coca plant. Afterwards, the coca plant is packed and shipped. At a storage facility, the coca plant is identified, prior to be sold to a costumer. A sample from the coca plant is taken, including the DNA oligomers (coding strands 110 and quantification strands 112). The sample is submerged in a suitable buffer solution, between about 1 and about 20 min. Then the mixture is deposited in a lateral flow test strip. As a result of the test a barcode may be generated. The barcode is read using a smartphone and is compared with a secure database of allowed results. The coca plant is successfully identified and sold.

In example #4 a cargo of opium poppy is encoded by depositing about 5 μL of encoding solution 108 onto the surface of the opium poppy. After harvest, the opium poppy is sprayed with a suitable encoding solution 108 including DNA oligomers, using a dispensing device 100, and left to dry. The encoding solution 108 used includes Alexa Fluor 647 fluorescent dye. A laser module 116 with a wavelength of about 633 nm is used as part of the feedback module 106. When laser module 116 illuminates the sprayed area, the dye particles are excited and emit fluorescent light of about 665 nm of wavelength. The intensity of the fluorescent light is measured by the optical detection module and it is determined that the amount of DNA oligomers deposited on the opium poppy is sufficient for the later identification of the opium poppy. Afterwards, the opium poppy is packed and shipped. At a storage facility, the opium poppy is identified, prior to be sold to a costumer. A sample from the plant is taken, including the DNA oligomers (coding strands 110 and quantification strands 112). The sample is submerged in a suitable buffer solution, between about 1 and about 20 min. Then the mixture is deposited in a lateral flow test strip. As a result of the test a barcode may be generated. The barcode is read using a smartphone and is compared with a secure database of allowed results. The opium poppy is successfully identified and sold.

In example #5 a cargo of genetically enhanced seeds is encoded by depositing about 5 μL of encoding solution 108 onto a sample of the seeds. After harvest, the seeds are sprayed with a suitable encoding solution 108 including DNA oligomers, using a dispensing device 100, and left to dry. The encoding solution 108 used includes Alexa Fluor 647 fluorescent dye. A laser module 116 with a wavelength of about 633 nm is used as part of the feedback module 106. When laser module 116 illuminates the sprayed area, the dye particles are excited and emit fluorescent light of about 665 nm of wavelength. The intensity of the fluorescent light is measured by the optical detection module and it is determined that the amount of DNA oligomers deposited on the plant is sufficient for the later identification of the seeds. Afterwards, the encoded seeds are packed and shipped with the rest of the seeds. At a storage facility, the seeds are identified, prior to be sold to a costumer. The encoded seeds are taken, including the DNA oligomers (coding strands 110 and quantification strands 112). The sample is submerged in a suitable buffer solution, between about 1 and about 20 min. Then the mixture is deposited in a lateral flow test strip. As a result of the test a barcode may be generated. The barcode is read using a smartphone and is compared with a secure database of allowed results. The seeds are successfully identified and sold.

While various aspects and embodiments have been disclosed, other aspects and embodiments are contemplated. The various aspects and embodiments disclosed are for purposes of illustration and are not intended to be limiting, with the true scope and spirit being indicated by the following claims.

The embodiments described above are intended to be exemplary. One skilled in the art recognizes that numerous alternative components and embodiments that may be substituted for the particular examples described herein and still fall within the scope of the invention.

What is claimed is:

1. A dispensing device comprising:
a DNA reservoir configured to store encoding solution, wherein the encoding solution comprises coding strands of DNA that encode a characteristic about a plant and quantification strands of DNA comprising synthetic DNA oligomers conjugated with fluorescent dye molecules; and
a spraying device configured to dispense the encoding solution on the plant; whereby the encoding solution is illuminated by a laser beam.

2. The dispensing device of claim 1, further comprising a feedback device comprising:
a laser beam generator configured to generate a laser beam that excites the fluorescent dye molecules and cause the fluorescent dye molecules to emit fluorescent light, wherein an intensity of fluorescent light emitted by the fluorescent dye molecules is directly proportional to an amount of coding strands of DNA deposited on the plant; and
an optical light detector configured to measure the intensity of the fluorescent light emitted by the fluorescent molecules and determine whether the intensity of the fluorescent light exceeds a first threshold, thereby determining whether the amount of coding strands of DNA deposited on the plant exceeds a second threshold.

3. The dispensing device of claim 1, wherein the feedback device further comprises:
a screen configured to indicate to an operator of the dispensing device whether more encoding solution should be deposited onto the plant in response to a non-transient signal from the optical light detector.

4. The dispensing device of claim 1, wherein the spraying device is a nozzle.

5. The dispensing device of claim 1, wherein the fluorescent dye molecules comprise Alexa Fluor® 647, Cy5, or DyLight 633 fluorescent dye.

6. The dispensing device of claim 1, wherein the fluorescent dye molecules conjugate with the DNA oligomers of the quantification strands between the 5′ and 3′ terminal ends of the DNA oligomers.

7. The dispensing device of claim 1, wherein the feedback device further comprises:
a battery powering the laser beam generator and the optical light detector.

8. The dispensing device of claim 1, wherein the laser beam has a wavelength between 405 and 633 nm.

9. The dispensing device of claim 1, wherein the laser beam generator further comprises a steering optic configured to direct the laser beam to converge at a focal point of the optical light detector.

10. The dispensing device of claim 1, wherein the laser beam generator assists the operator in aiming the spraying device.

11. The dispensing device of claim 1, wherein the optical detection unit further comprises:
a photodetector configured to receive and analyze light;
a collection and focusing optic configured to collect light at a focal point of the focusing optic and refocus the collected light into the photodetector; and
an optical filter configured to filter out laser light from the light collected by the collection and focusing optic but allow the fluorescent light collected by the collection and focusing optic to pass through to the photodetector.

12. The dispensing device of claim 11, wherein the optical filter is a bandpass filter or a notch filter.

13. The dispensing device of claim 11, wherein the collection and focusing optic is protected by a splash guard to avoid optical interference.

14. The dispensing device of claim 1, wherein the plant is a cannabis plant.

15. A method for measuring a quantity of a solution deposited on a plant comprising:
focusing a laser beam, by a steering optic, at a part of the plant where an encoding solution has been deposited to excite the fluorescent dye molecules and cause the fluorescent dye molecules to emit fluorescent light,
wherein the encoding solution comprises coding strands of DNA that encode a characteristic about the plant and quantification strands of DNA comprising synthetic DNA oligomers conjugated with fluorescent dye molecules, and
wherein an intensity of fluorescent light emitted by the fluorescent dye molecules is directly proportional to an amount of coding strands of DNA deposited on the plant;
collecting light by a collection and focusing optic;
receiving the collected light by a photodetector;
measuring the intensity of the fluorescent light by the photodetector; and
determining whether the intensity of the received fluorescent light exceeds a threshold by the photodetector, thereby determining whether a sufficient amount of coding strands of DNA have been deposited on the plant.

16. The method of claim 15, further comprising:
filtering the collected light using an optical filter to filter the laser light while allowing the fluorescent light to pass through the optical filter after the collection and focusing optic collects the light.

17. The method of claim 15, further comprising:
indicating to an operator of dispensing device, using a screen of the dispensing device, whether the intensity of the received fluorescent light exceeds the threshold.

18. The method of claim 15, wherein the laser beam generator assists the operator in aiming the spraying device.

19. The method of claim 15, wherein the laser beam has a wavelength between 405 and 633 nm.

20. The method of claim 15, wherein the plant is a cannabis plant.
